(19) United States
(12) Patent Application Publication
(10) Pub. No.: US 2024/0189428 A1
(43) Pub. Date: Jun. 13, 2024

(54) IMPLANTABLE OR INJECTABLE PRODUCTS BASED ON POLYMERS AND METHOD FOR THEIR PREPARATION

(71) Applicant: SYMATESE, Chaponost (FR)

(72) Inventors: Fabien FUCHEZ, Montrottier (FR); Chantal BELIN, Vienne (FR); Benjamin HERBAGE, Lyon (FR)

(73) Assignee: SYMATESE, Chaponost (FR)

(21) Appl. No.: 18/548,300

(22) PCT Filed: Mar. 2, 2022
(86) PCT No.: PCT/EP2022/055296

§ 371 Date: Aug. 29, 2023

(30) Foreign Application Priority Data
Mar. 2, 2021 (EP) 21305249.1

(51) Int. Cl. A61K 47/36; A61K 9/00; A61K 31/167; A61L 27/20; A61L 27/26; A61L 27/52; A61L 27/54; C08B 37/08; C08J 3/24; C08L 5/08

(52) U.S. Cl. CPC A61K 47/36; A61K 9/0024; A61K 31/167; A61L 27/20; A61L 27/26; A61L 27/52; A61L 27/54; C08B 37/0072; C08J 3/24; C08L 5/08; A61L 2300/402; A61L 2400/06; A61L 2430/34; A61L 2430/40; C08J 2305/08

(57) ABSTRACT

The present invention concerns a method for the preparation of a product based on a crosslinked polymer, comprising a step for crosslinking said polymer or one of its physiologically acceptable salts in the presence of a crosslinking agent comprising at least two epoxy functions, said crosslinking being carried out at a temperature of more than 0° C. and less than 10° C., as well as the implantable or injectable products obtained therefrom, advantageously based on hyaluronic acid.

### TECHNICAL FIELD

The invention relates to a method for crosslinking polymers such as hyaluronic acid, enabling implantable or injectable products to be produced.

It also concerns the implantable or injectable products obtained in this manner, having remarkable properties. They have a number of applications, both in the field of aesthetics (wrinkle fillers, breast enlargement, etc), in plastic surgery (scar repair) and in the therapeutic field (viscosupplementation agent for joints, or tissue spacers, for example in urology or in the treatment of cancer by radiotherapy).

### BACKGROUND

Products based on crosslinked polymers have now been used in routine, both in the aesthetics field and in the medical field, for applications as diverse as filling wrinkles and the treatment of osteoarthritis.

Wrinkle fillers based on hyaluronic acid constitute a good example of the technical problems generally encountered with products based on crosslinked polymers.

Hyaluronic acid (HA) is a glycosaminoglycan (or GAG) which is essentially found in the extracellular matrix of connective tissue known as soft tissue, such as the skin (in contrast to other tissue known as hard tissue, such as cartilage). In the skin, hyaluronic acid is essentially synthesized by dermal fibroblasts and epidermal keratinocytes, but it is physiologically degraded by hyaluronidases. With age, the quantity of hyaluronic acid which is synthesized and its degree of polymerisation decrease, contributing to the visible signs of skin ageing.

Hyaluronic acid is a polymer constituted by repeated disaccharide units and is naturally present in human connective tissue (hence the qualification as a biopolymer), and which is also resorbable, i.e. capable of being degraded in vivo. Furthermore, it has the property of capturing and retaining molecules of water. When it is hydrated, it naturally forms a viscous composition with the appearance of a gel.

Because of these characteristics, hyaluronic acid is a polymer particularly suitable for the production of wrinkle filling products, also known as “fillers”, which are essentially intended to be injected. However, in order to improve the mechanical properties and the in vivo life span thereof, at least a portion of the hyaluronic acid contained in these products is usually crosslinked, for example chemically with the aid of a crosslinking agent.

The crosslinking reaction has the effect of generating a network comprising molecules of hyaluronic acid linked together via molecules of the crosslinking agent. The polymer network which is formed generates an aqueous gel. The more crosslinked the polymer, the more viscous is the resulting gel, and the harder it is and the more resistant to in vivo degradation. However, in the case of excessive crosslinking, the gel could become brittle and fragment, or generate inflammation after implantation or injection, leading to its enzymatic degradation.

Furthermore, since products of this type are mainly intended to be injected, the gel has to be sufficiently fluid to allow it to flow in the injection device and prevent it from clogging.

Thus, technical solutions have been investigated in order to obtain products that satisfy these various requirements.

In practice, it is possible to modulate the degree of crosslinking of the polymer by varying certain parameters of the crosslinking reaction. The crosslinking degree corresponds to the ratio of the number of moles of disaccharides of the polymer which are effectively crosslinked over the total number of moles of disaccharides of the polymer.

Furthermore, in products in the form of aqueous gels and comprising the crosslinked polymer, a free polymer, i.e. which is not crosslinked, is sometimes added. Adding free polymer can then be used to reduce the viscosity of the product and facilitate application by injection, generally carried out with the aid of a syringe.

In the frame of aesthetics applications in particular, it is beneficial to have products available which have sufficient properties of hardness and resistance to compression. Hardness is closely linked to the capacity of solids or gels to resist an elastic deformation stress, conventionally measured by means of the modulus G′. The strength to resist linear compression can be used to describe the behaviour of a solid or gel which is subjected to a linear compressive type stress, e.g. a uniaxial stress. It is generally considered that the materials which have a high strength to resist linear compression have good cohesion or cohesivity, i.e. the molecules of which it is composed are linked together by large forces, which means that they can avoid disintegration or diffusion of the product under the effect of pressure applied in a uniaxial manner.

Thus, once injected, the product is capable of resisting the various forces which are exerted on it without rupturing or diffusing from the zone to be filled. Properties of this type are typically obtained by increasing the crosslinking degree of the hyaluronic acid present in the injectable product, with the possible disadvantages mentioned above.

Furthermore, in addition to the linkages described above, the crosslinking reaction may produce chemical modifications which do not participate in true crosslinking the molecules of hyaluronic acid, but in modifying its structure. This is the case, for example, with formation of covalent bonds between a molecule of crosslinking agent and a single molecule of hyaluronic acid (referred to as “grafting”). As used herein, modifications induced by the crosslinking agent are generally designated by the term “modifications”, irrespective of whether the modifications participate in true crosslinking or other modifications such as grafting. The extent of such modifications due to the crosslinking reaction may be estimated by the degree of modification (“MOD”). In general, the MoD of an injectable product comprising a crosslinked disaccharide corresponds to the percentage of the number of moles of crosslinking agent which are bonded in any manner with respect to the number of moles of disaccharide units forming the polymer. On the one hand, this parameter takes into account the molecules of crosslinking agent actually participating in crosslinking, i.e. bonded between two disaccharide molecules. On the other hand, it also takes into account the molecules of crosslinking agent linked to a single molecule of polymer, such as by grafting, and not participating in crosslinking of the polymer.

However, it has been suggested that a too high degree of modification could affect the biocompatibility of the crosslinked hyaluronic acid (A. Tezel, G.H. Fredrickson, Journal of Cosmetic and Laser Therapy, 10(1):35-42, 2008). Thus, highly modified hyaluronic acids are now suspected of being the source of a certain number of secondary reactions (allergic reactions, inflammatory reactions, or even rejection). Thus, this constitutes an additional disadvantage to the fragmentation observed for highly crosslinked products.

A further disadvantage of highly crosslinked fragmented products is that they cannot be used as viscosupplementation agents, e.g. in the treatment of osteoarthritis: For said application, the filler has to be hydrophilic in order to be able to homogeneously swell in the joint and properly act as a lubricant.

Thus, there is a need for the development of injectable products with interesting properties, in particular rheological properties, but which do not suffer from the disadvantages of the highly modified hyaluronic acid gels which are currently available.

### DETAILED DESCRIPTION

In order to satisfy all of these demands, the Applicant has developed a novel crosslinking method which can be integrated into a method for the preparation of implantable or injectable products containing a crosslinked polymer.

According to the invention, crosslinking of said polymer is carried out at a low temperature with the aid of a crosslinking agent.

It is generally known that the crosslinking step is facilitated by high temperatures. Surprisingly and unexpectedly, however, the Applicant has discovered that when crosslinking is carried out at a low temperature, effective crosslinking can be achieved, leading to the production of products which effectively have the qualities of an aqueous gel, in particular in terms of rheology. Furthermore, and unexpectedly, this method can be used to produce products having excellent mechanical properties, in particular a high strength to resist linear compression even for low degrees of modification. Besides, the crosslinking step performed at low temperature has been shown to prevent the degradation of the polymer chains, giving rise to a final product having inter alia a reduced quantity of soluble polymer as well as a better resistance especially to thermal stress.

As a consequence, these gels are sufficiently resistant to pressure to be used under circumstances which usually require aqueous gels of highly crosslinked polymers, without suffering from the disadvantages thereof.

The injectable products of the invention also have good temperature stability. In fact, it has been shown that their rheology properties are relatively well preserved, even after several days at high temperatures.

Finally, the injectable products prepared according to the methods described herein have the additional feature that the great majority of the crosslinked hyaluronic acid contained therein is in the form of networks of crosslinked polymer of large size. As a consequence, the products of the invention contain few hyaluronic acid networks of small size, thereby guaranteeing a good filling or lubricating effect and being less susceptible to in vivo degradation, which ensures that they will perform well over long periods.

Thus, the present application reports a method for the preparation of products comprising a crosslinked polymer, the products obtained by said method, and more generally implantable or injectable products having the features of such products.

The invention then concerns a method for the preparation of products based on a crosslinked polymer, the method comprising at least one step for crosslinking said polymer or one of its physiologically acceptable salts, in the presence of a crosslinking agent, said crosslinking being carried out at a low temperature, for example in the range of about 0° C. to about 10° C.

According to a first aspect, the invention concerns a method of preparing a product containing a crosslinked polymer, wherein the crosslinking step comprises or consists in incubating in a reaction mixture the polymer, or one of its physiologically acceptable salts, with a crosslinking agent having advantageously at least two epoxy functions, at a low temperature, advantageously of more than 0° C. and less than 10° C., more advantageously between 1° C. and 9° C.

The method of the invention is thus carried out on a polymer of interest in the context of the envisaged applications, in particular those routinely used in medical devices. A polymer is defined as a succession of monomeric units polymerised together, forming a simple chain. Appropriately, the polymer has free functions which are capable of reacting with the crosslinking agent, advantageously alcohol or amine functions.

According to one aspect, the polymer used or implemented in the method of the invention is in its native form, also termed in the context of the present invention a “non-crosslinked polymer” or a “free polymer”, namely a polymer which is not crosslinked or not modified, i.e. in which the chains are not bridged together or grafted with molecules of crosslinking agent.

Alternatively, the method of the invention may be carried out on a polymer which has already been partially reacted with a crosslinking agent.

Advantageously, the polymer implemented is a biocompatible polymer, also termed a “biopolymer”, naturally present in the organism into which it is intended to be injected.

In accordance with another particular aspect, said polymer has the advantage of being resorbable in vivo, i.e. it is capable of being naturally degraded by the organism into which it is intended to be implanted or injected. In a known manner, this property may be tested in vitro in the presence of an enzyme which is capable of degrading it (e.g. in the presence of a hyaluronidase in the case of hyaluronic acid) or in vivo (in an animal model or in a human being) by measuring the time necessary for its volume to be reduced, or even for it to disappear. Appropriately, the product obtained in the context of the invention has a resorption time which may be from several weeks to several months.

In accordance with a preferred embodiment, the polymer implemented is selected from the group comprising polysaccharides, collagen and elastin.

Preferably, the polysaccharides used in the context of the invention include glycosaminoglycans (GAG), alginate, dextran, chitosan and cellulose.

Advantageously, the polymer implemented belongs to the glycosaminoglycan family (GAG). Within the meaning of the invention, the term “glycosaminoglycans” designates unbranched polysaccharide polymers composed of repeated disaccharides always containing a hexosamine (glucosamine (GlcN) or galactosamine (GalN)) and another ose (glucuronic acid (GlcA), iduronic acid (IdoA), galactose (Gal)). The glucosamine is either N-sulphated (GlcNS), or N-acetylated (GlcNac). The galactosamine is always N-acetylated (GalNac). Advantageously, the polymer which is a glycoaminoglycan is selected from the group comprising hyaluronic acid, chondroitin sulphate, dermatan sulphate, keratan sulphate, heparin or heparan sulphate.

A particularly suitable polymer in the context of the invention is hyaluronic acid. Within the meaning of the invention, the term “hyaluronic acid” designates a polymer of molecules of hyalobiuronic acid, otherwise known as ß-D-glucuropyrannosyl (1→3)-ß-D-2-N-acetylglucosaminic acid (CAS number: 499-15-0), bonded together by beta-1,4 glycoside bonds. Hyalobiuronic acid comprises one molecule of D-glucuronic acid and one molecule of D-N-acetylglucosamine, bonded together via alternating ß-1,4 and ß-1,3 glycosidic bonds.

Within the meaning of the invention, a “physiologically acceptable salt” may, without limitation, be chosen from sodium, potassium, zinc or silver salts. Thus, the term “physiologically acceptable hyaluronic acid salt” in certain embodiments designates sodium hyaluronate, potassium hyaluronate, zinc hyaluronate, silver hyaluronate and their mixtures, preferably sodium hyaluronate.

The method of the invention can be implemented with a polymer of any molecular weight, in particular hyaluronic acid with any molecular weight. However, and advantageously, in the case of hyaluronic acid, this has an average molecular weight of less than or equal to 1×10⁷ Daltons (Da). Advantageously, the hyaluronic acid used in the method of the invention has an average molecular weight of from 5.0×10⁴ Da to 1×10⁷ Da, advantageously in the range 1×10⁵ Da to 5.0×10⁶ Da.

The polymer used in the context of the invention can readily be obtained industrially using methods which are well known to the person skilled in the art. Thus, and by way of example, the hyaluronic acid may be obtained by extraction from animal tissue (such as rooster combs) or by bacterial fermentation.

In accordance with a further characteristic, the method in accordance with the invention employs a crosslinking agent or crosslinker which is capable of inducing crosslinking, i.e. bridging, between different chains of polymer.

In accordance with a particular embodiment, such a compound comprises epoxy functions. These functions are capable of reacting with the polymer which is present, advantageously with the alcohol or amine functions thereof. According to a particular embodiment, such a compound comprises at least two epoxy functions, advantageously two epoxy functions (“bis-epoxide”), yet more advantageously located at each chain end, i.e. one epoxy function at each chain end of the compound. In accordance with a particular embodiment, the crosslinking agent is selected from poly(ethylene glycol) diglycidyl ethers, also known as PEG diglycidyl ether. The crosslinking agent is preferably selected from the following group: 1,4-butanediol diglycidyl ether (BDDE, CAS number: 2425-79-8), ethylene glycol diglycidyl ether (EGDE, CAS number: 2224-15-9), diethylene glycol diglycidyl ether (CAS number: 4206-61-5), and tetraethylene glycol diglycidyl ether. In accordance with a particular embodiment, the crosslinking agent is 1,4-butanediol diglycidyl ether.

In accordance with another particular embodiment, the crosslinking agent that is used in the method is preferably selected from PEG diglycidyl ethers wherein the poly(ethylene glycol) chain does not contain more than 3 or even 2 oxygen atoms. The crosslinking agent is preferably selected from the group consisting of 1,4-butanediol diglycidyl ether, ethylene glycol diglycidyl ether, and diethylene glycol diglycidyl ether, advantageously from the group consisting of 1,4-butanediol diglycidyl ether and ethylene glycol diglycidyl, more advantageously 1,4-butanediol diglycidyl ether.

The crosslinking reaction may, for example, consist of mixing or incubating the polymer and the crosslinking agent in the temperature range of about 0° C. to about 10° C., or of about 1° C. to about 9° C.

During this reaction, the polymer will then be modified by the crosslinking agent. In the context of the invention, the “modified polymer” designates a polymer comprising structural modifications obtained from a crosslinking step, including crosslinking bonds between two polymer molecules via a molecule of the crosslinking agent (hereinafter designated “crosslinking linkages”), as well as any other types of linkages such as, for example, linkages between a single molecule of polymer and a molecule of crosslinking agent. Although these latter do not participate in crosslinking of the polymer, they constitute structural modifications. Thus, within the meaning of the invention, the structural modifications obtained from a crosslinking step include both crosslinking linkages and other types of modifications which are susceptible of being induced by the crosslinking reaction, e.g. the grafting of molecules of crosslinking agent onto the polymer. Thus, within the meaning of the invention, the concentration, or, if appropriate, the proportion of modified polymer, corresponds to the concentration or the proportion of polymer comprising structural modifications obtained from a crosslinking step.

In accordance with one embodiment, the polymer undergoing the crosslinking step is in solid form, for example as fibres or as a powder. Advantageously, and for the crosslinking reaction, the polymer is mixed with a liquid phase, this step possibly being carried out prior to or concomitantly with the crosslinking reaction. This step is easily carried out by mixing the polymer with a liquid phase, for example an aqueous solution. In the context of the invention, an aqueous solution may be constituted by water or comprise water and one or more other solvents such as dimethylsulphoxide (DMSO).

Thus, the polymer or one of its physiologically acceptable salts, in the non-crosslinked form, is hydrated in an aqueous medium, then mixed with the crosslinking agent. Alternatively, the polymer or one of its physiologically acceptable salts, in the non-crosslinked form, is mixed with an aqueous solution of the crosslinking agent.

According to a particular embodiment, the crosslinking reaction is carried out in a basic medium, preferably at a pH of more than 7, or even greater than or equal to 8, 9 or even 10. A basic pH promotes the crosslinking reaction. The basic pH may be obtained by using basic solutions which are in routine use in this field, such as sodium hydroxide, for example.

During the crosslinking step of the method of the invention, the reaction mixture therefore corresponds to a basic preparation comprising the polymer and the crosslinking agent.

The polymer concentration in the reaction mixture is adjusted by the person skilled in the art. The increase in the polymer concentration promotes the crosslinking reaction and can be used to reduce the quantity of crosslinking agent. However, it should be compatible with good hydration of the polymer and good homogenization of the reaction medium. In relation to hyaluronic acid, for example, its concentration in the reaction mixture may be in the range 50 to 500 mg per gram of reaction mixture, for example between 100 and 200 mg per gram of reaction mixture.

Furthermore, the skilled person may in particular vary the respective quantities of crosslinking agent and polymer in the reaction mixture. Thus, and appropriately, the molar ratio between the crosslinking agent and the disaccharide units of the polymer is greater than or equal to about 0.5%. Below this ratio, the final product may be too liquid for the envisaged applications. In accordance with another embodiment, this ratio does not exceed about 10%. In one embodiment, the molar ratio ranges from 0.5% to 10%. Above this ratio, the final product obtained from the claimed method may risk being too brittle. The term “disaccharide units of polymer” as used within the meaning of the invention should be understood to mean repeated disaccharide units forming the polymer as defined above. By way of example and as already stated, hyaluronic acid is formed from repeated units of hyalobiuronic acid, which is a disaccharide which itself is formed from D-glucuronic acid and D-N-acetylglucosamine.

In order to facilitate a uniform reaction, the mixture may advantageously be homogenized, e.g. mechanically, with the aid of any method which is suitable and known to the person skilled in the art.

Characteristically, in accordance with the invention, the crosslinking step is carried out at low temperature. This temperature is advantageously more than 0° C., or even 1, 2, 3, 4 or 5° C. Preferably, it is below 10° C., or even 9, 8, 7 or 6° C. In certain embodiments, the temperature ranges from greater than about 0° C. to less than about 10° C., such as greater than about 0° C. to less than about 9° C., greater than about 0° C. to less than about 8° C., greater than about 0° C. to less than about 7° C., greater than about 0° C. to less than about 6° C., greater than about 0° C. to less than about 5° C., greater than about 1° C. to less than about 10° C., greater than about 1° C. to less than about 9° C., greater than about 1° C. to less than about 8° C., greater than about 1° C. to less than about 7° C., greater than about 1° C. to less than about 6° C., greater than about 1° C. to less than about 5° C., greater than about 2° C. to less than about 10° C., greater than about 2° C. to less than about 9° C., greater than about 2° C. to less than about 8° C., greater than about 2° C. to less than about 7° C., greater than about 2° C. to less than about 6° C., greater than about 2° C. to less than about 5° C., greater than about 3° C. to less than about 10° C., greater than about 3° C. to less than about 9° C., greater than about 3° C. to less than about 8° C., greater than about 3° C. to less than about 7° C., greater than about 3° C. to less than about 6° C., or greater than about 3° C. to less than about 5° C. In other embodiments, the temperature may be about 0° C., about 1° C., about 2° C., about 3° C., about 4° C., about 5° C., about 6° C., about 7° C., about 8° C., about 9° C., or about 10° C. In practice, the reaction mixture is advantageously placed in a vessel at the desired temperature, enabling the temperature to be maintained throughout the duration of the crosslinking reaction.

Preferably, the crosslinking step is carried out for a period of at least or more than 24 or 48 hours, advantageously greater than or equal to 7, 8, 9, 10, 11, 12, 13 or even 14 days, possibly up to one or more weeks. Preferably, the desired temperature is maintained substantially constant throughout the duration of the crosslinking reaction.

At the end of the crosslinking reaction, the product so obtained is advantageously in the form of a gel of modified polymer. In practice, proper execution of the crosslinking step results in an increase in the viscosity of the reaction mixture.

At the end of this crosslinking step, the reaction mixture may undergo a certain number of subsequent treatments that may be carried out sequentially or simultaneously, in the order indicated or in a different order:

One method in accordance with the invention may optionally comprise a step for neutralization, which can reduce the pH to a value below about 8, advantageously to bring it to an approximately neutral pH, typically greater than or equal to 6.5, or even 6.6, 6.7, 6.8 or 6.9, and less than or equal to 7.5, or even 7.4, 7.3, 7.2, 7.1 or 7. This drop in pH can be used to stop the crosslinking reaction, ensuring better stability of the polymer that is present, or to place the product at a pH which is compatible with the envisaged applications, in particular with that of bodily fluids or the skin. This step may, for example, be carried out by mixing the product obtained from the crosslinking step with a composition with an acidic pH, such as a solution of hydrochloric acid (HCl), for example. In fact, this step for neutralization may be accompanied by a dilution of the mixture obtained from the crosslinking reaction.

The preparation method in accordance with the invention may also comprise a step for dilution or hydration of the product obtained from the crosslinking reaction. This may be carried out, for example, by adding a buffer solution with a suitable pH to the preparation, advantageously a neutral pH in the range 6.5 to 7.5, e.g. a solution of phosphate buffer (PBS, for “Phosphate Buffered Saline”). The dilution factor may typically vary between about 2 and 10, for example between about 4 and 8 or about 5.

It should be noted that the steps for neutralization and dilution may optionally be carried out concomitantly, using a suitable solution which can be used to obtain the desired pH and concentration in particular of crosslinked hyaluronic acid.

In accordance with another aspect, the preparation method of the invention may also comprise a step for purification of the product obtained at the end of the crosslinking step, in particular in order to ensure that the crosslinking agent which has not reacted with the polymer during the crosslinking step, and possible other impurities, are eliminated. Advantageously, this treatment is carried out after the neutralization and/or the dilution. This step can readily be carried out by the person skilled in the art using any appropriate known method for elimination of unwanted residue, e.g. residual crosslinking agent. It may, for example, be carried out by means of successive washes.

In accordance with a particular embodiment, in addition to the crosslinking step, the method in accordance with the invention may comprise:
a step for neutralization, preferably carried out with the aid of an acidic solution, and/or
a step for dilution, preferably carried out with the aid of a neutral solution; and/or;
a step for purification, for example by washing.

In accordance with one particular embodiment, the method comprises all of these steps carried out in this order. However, and by way of example, the steps of neutralization and of dilution may be concomitant.

Furthermore, the composition of the product obtained at the end of the method in accordance with the invention may be modified:

A free (unmodified) polymer, or one of its physiologically acceptable salts, may be added to the product obtained in this manner. This step has the effect of reducing the viscosity of the product, which may prove to be useful in the context of injectable preparations. Advantageously, the same polymer as that used for the crosslinking step, preferably with substantially the same molecular weight, may be added.

Thus, for example when hyaluronic acid is used, a phosphate buffer solution comprising hyaluronic acid, advantageously sodium hyaluronate, may be added. The concentration and the volume of this solution may be adjusted in a manner such as to obtain the desired concentration of free polymer in the final product, advantageously in the range of about 0.5 to 5 mg per gram of final product in the case of hyaluronic acid.

In view of the envisaged applications, it may also be advantageous to add anaesthetic compounds such as, for example, lidocaine, bupivacaine, mepivacaine, etc., which have the effect of limiting the pain linked to injection. A final concentration of anaesthetic in the range about 1 to about 5 mg per gram, such as about 2 to about 4 mg per gram or about 3 mg per gram, of final product is conventionally employed. According to a preferred embodiment, the addition of the anaesthetic compound, e.g. lidocaine, does not require any pH adjustment prior to its introduction into the composition.

Alternatively or in addition, other active ingredients may be added to the products obtained at the end of the method in accordance with the invention. It may, for example, be a vasoconstrictor which reduces secondary effects linked to injection, or any active ingredient of interest in the context of the envisaged application, for example an antioxidant, in particular mannitol, or an anti-inflammatory agent. The addition of active ingredients of this type in a suitable concentration is within the knowledge of the person skilled in the art.

By way of example, the product obtained at the end of the various steps of the method in accordance with the invention may also be formulated:
by adding polymer or one of its physiologically acceptable salts in the non-crosslinked form, and/or
by adding an anaesthetic agent, for example lidocaine, and more generally any active ingredient of interest.

With each of these additions, it is recommended that the volumes should be adjusted in order to maintain the desired concentrations of polymer. These various additions may be carried out simultaneously or successively. Advantageously, the mixture may be homogenized after each addition.

Furthermore and preferably, the product obtained at the end of the various steps of the method of the invention is sterilized. Sterilization may be carried out using any of the usual suitable methods such as, for example, sterilization by heat treatment, in an autoclave. The sterilization temperature is advantageously in the range 100° C. to 150° C., preferably more than 121° C.

A product so obtained is advantageously intended to be implanted into the body, preferably into the human body. An appropriate manner of implanting a product of this type at an implantation site of interest consists of injecting it into the body, advantageously with the aid of an injection device such as a syringe. Thus, and in accordance with another aspect, the product obtained with the aid of the method of the invention is packaged into one or more syringes, with a volume which is adapted to the envisaged application. Sterilization may be carried out before or after packaging.

The invention also concerns implantable or injectable products obtained by the methods described herein. In one particular embodiment, the present invention concerns implantable or injectable products obtained when the method described herein is carried out with hyaluronic acid or a salt thereof as the polymer.

Thus, in accordance with a preferred aspect, the invention concerns an implantable or injectable product which is capable of being obtained by the methods described above, and in particular relates to hyaluronic acid or one of its salts, in particular sodium hyaluronate, as a particularly preferred polymer. However, the teaching of the present invention also applies to any polymer, especially those containing disaccharide units.

A product prepared according to methods described herein may therefore comprise hyaluronic acid modified with the aid of a crosslinking agent carrying at least two epoxy functions, advantageously comprising an epoxy function at each of its ends, more advantageously chosen from 1,4-butanediol diglycidyl ether, ethylene glycol diglycidyl ether, diethylene glycol diglycidyl ether, or tetraethylene glycol diglycidyl ether.

Thus, the modified hyaluronic acid in accordance with the invention may comprise the following different structures:
chains of hyaluronic acid bridged together by means of the crosslinking agent;
chains of hyaluronic acid grafted by the crosslinking agent;
chains of hyaluronic acid which are both bridged and grafted.

Where the crosslinking agent is 1,4-butanediol diglycidyl ether, ethylene glycol diglycidyl ether, diethylene glycol diglycidyl ether, or tetraethylene glycol diglycidyl ether, the modified hyaluronic acid present in the product of the invention has chains which are grafted and/or bridged with the aid of said agent, as diagrammatically represented below:

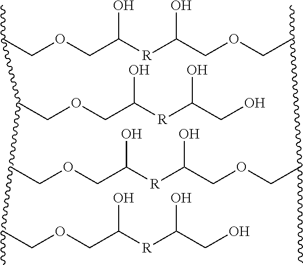

Preferably, and with respect to the envisaged applications, the modified or crosslinked hyaluronic acid represents about 1 to about 40 mg/g of the product according to the invention, advantageously about 5 to about 40 mg/g of product, more advantageously about 10 to about 25 mg/g of product.

Said product may furthermore comprise:
unmodified (non-crosslinked or free) hyaluronic acid, advantageously in a quantity of about 0.5 to about 5 mg/g of product, advantageously in a quantity of about 1 to about 3 mg/g of product; and/or
an anaesthetic agent, advantageously lidocaine; preferably in a quantity of about 1 mg to about 5 mg per gram of product, preferably about 3 mg per gram of product; and/or
any other suitable active ingredient, for example an antioxidant such as mannitol or an anti-inflammatory agent.

In accordance with a particular embodiment, the product does not contain any “free” crosslinking agent, i.e. crosslinking agent not grafted or not bridged to the polymer.

Appropriately, the product of the invention has a neutral or physiological pH, advantageously in the range of about 6 to about 8, more advantageously in the range 6.5 to 7.5. According to a particular embodiment, the pH of the product according to the invention is greater than or equal to 6.5, or even 6.6, 6.7, 6.8 or 6.9, and less than or equal to 7.5, or even 7.4, 7.3, 7.2, 7.1 or 7, e.g. in the range of 6.8 to 7.2.

Characteristically, in accordance with the invention, the product is in the form of a gel.

In accordance with another characteristic, said gel is an aqueous gel, i.e. comprising water. In accordance with a preferred embodiment, said gel is injectable, having a viscosity which is adapted to injection systems which are in routine use in the fields of the applications, in particular in syringes.

Gels are conventionally defined as viscous compositions which, however, behave like solids. The physical behaviour of gels is measured and expressed by the elastic modulus G′ and the viscous modulus G″.

A suitable measure of the solid behaviour of a gel is its resistance to deformation, which differs from that of liquids. In fact, when a deformation is applied to a solid body, it often responds by a strain which is proportional to the imposed deformation, which can be measured with the aid of the elastic modulus G′.

The elastic modulus G′ is the resultant of stress over deformation or strain (G′=stress/deformation), therefore describing the energy absorbed by a solid. The higher G′ is, the harder is the material. The elastic modulus G′ is conventionally used to describe the solid component of the gel. By way of example, in terms of orders of magnitude, it is generally considered that emulsions have an elastic modulus G′ of the order of a Pascal, while hard solids have an elastic modulus G′ of the order of a giga Pascal.

Therefore, “gel” as used in the context of the invention advantageously means a composition having an elastic modulus G′ of more than 10 Pa. Preferably, the products of the invention have an elastic modulus G′ in the range of about 20 to about 1000 Pa. According to a preferred embodiment, a product of the invention displays an elastic modulus greater than about 20, 30, 40 or even 50 Pa. According to another preferred embodiment, a product of the invention displays an elastic modulus less than about 1000, 900, 800, 700, 600 or even 500 Pa. Advantageously, the G′ value of the products of the invention is in the range of about 50 to about 500 Pa. In the context of the present invention, the value G′ is correlated to the degree of crosslinking of the polymer, contributing to the solid component of the envisaged product.

The modulus G′ of the product of the invention is determined using methods which are well known to the person skilled in the art. In the frame of the invention, the modulus G′ is preferably determined at ambient temperature, typically at a temperature equal to 25° C., with the aid of a rheometer, for example of the Kinexus Pro type, using a 40 mm plate/plate geometry and a 250 μm gap. Conventionally, pre-shearing and a rest period are applied to the sample in order to erase its rheological memory, then the linear viscoelastic domain is determined by an amplitude sweep, for example at a fixed frequency of 1 Hz. The modulus G′ can then be determined from the curve which is obtained in the linear viscoelastic region where the stress and displacement are in a linear relationship.

In particular, the implantable or injectable products obtainable from the method of the invention have mechanical and structural properties which distinguish them from known products. In particular, the results from deformation resistance tests provide evidence that they have good cohesive properties, illustrated by a high strength to resist linear compression, even though their degree of modification is low. The implantable or injectable products of the invention are thus characterized by these parameters in particular.

In the context of the invention, the strength to resist linear compression (F or uniaxial compressive strength force) is measured when the product is compressed with the aid of a rheometer, for example of the Kinexus Pro type, as for the measurement of the modulus G′ above, with a 20 mm plate/plate geometry with a gap varying from 2.5 mm to 0.9 mm, at a speed of 0.1 mm/sec. This measurement is advantageously carried out at ambient temperature, typically at a temperature equal to 25° C. Thus, in the context of the invention, the term “strength to resist linear compression” designates the force expressed in Newtons obtained under the test conditions described above.

Advantageously, the products of the invention have a strength to resist linear compression (F) of at least about 0.15 Newton (N), advantageously of at least about 0.2, 0.25, 0.3, 0.35, 0.4, 0.45, 0.5, 0.55, 0.6, 0.65 or 0.7 N. According to another embodiment, the strength to resist linear compression (F) does not exceed about 1.6, 1.55, 1.5, 1.45, 1.4, 1.35, 1.3, 1.25, 1.2, 1.15, 1.1, 1.05 or 1 N. According to a specific embodiment, the products of the invention have a strength to resist linear compression in the range of about 0.2 N to about 1.2 N, or even in the range of about 0.35 N to about 1 N.

The products of the invention may also be characterized by their degree of modification or “MoD”, correlated with the modifications to the hyaluronic acid induced by the crosslinking agent.

As mentioned above, the degree of modification or “MoD” illustrates the biocompatibility of the product. The more modifications the product has, the more it is distinguished from the free or unmodified polymer, and the less biocompatible it is.

A technical definition of the degree of modification as well as a normalised method for measuring this degree of modification are detailed in document Kenne et al. (Carbohydrate Polymers; 91 (1): 410-4182, 2013), incorporated by reference herein, to which the skilled person may refer.

In the frame of the present invention, the term “degree of modification” designates the ratio between the number of moles of crosslinking agent fixed to the polymer and the number of moles of disaccharides of the polymer, as determined by Kenne et al.'s method. When hyaluronic acid is the polymer which is used, the ratio is then that of the number of moles of crosslinking agent fixed to the hyaluronic acid and the number of moles of disaccharides comprised in the hyaluronic acid, i.e. the number of moles of hyalobiuronic acid as defined above. This ratio is typically expressed in the form of a percentage.

In brief, the product is washed in a solution of sodium chloride then filtered under vacuum using a 0.22 μm filter, before being digested with chondroitinase AC at 37° C. overnight. The number of moles of crosslinking agent fixed to hyaluronic acid molecules and the number of moles of hyaluronic acid which are effectively crosslinked are determined using NMR (in particular ¹H or ²D if necessary), by integration of the signal corresponding to residues of the crosslinking agent and the signal corresponding to N-acetyl of hyaluronic acid after correction for the number of protons responsible for each signal.

Thus, and in accordance with the invention, the products preferably have a degree of modification (MoD) of less than about 15%, or even equal to or less than about 10%, 9%, 8%, 7%, 6%, 5.5%, 5%, 4.5%, 4%, 3.5%, 3%, 2.5%, 2%, 1.5%, 1% or 0.5%. According to a specific embodiment, the products of the invention have a degree of modification (MoD) of less than about 10%, advantageously between about 0.5% and about 6%.

Remarkably, for a given value of F, the products obtained have a particularly low degree of modification. Thus, the products of the invention can be characterized by the value F/MOD, corresponding to the absolute value of the ratio between the strength to resist linear compression as measured above (F) and the degree of modification (MoD) expressed as a percentage.

Advantageously, this ratio is greater than about 0.15, equal to or more than about 0.16, 0.17, 0.18, 0.19 or 0.2, even equal to or more than about 0.3, 0.4, 0.5, 0.6, 0.7 or 0.8. Furthermore, it may reach a value of about 0.9, or even of about 1. In other words, for a degree of modification (MoD) of about 1%, the strength to resist linear compression (as measured above) will be more than about 0.2 N.

Furthermore, in at least some embodiments, the injectable products of the invention are particularly resistant to temperature, possibly due to the preservation of the polymer chains during the crosslinking step performed at low temperature. In fact, and as will be demonstrated in the experimental section, when they are subjected to several days at a temperature of 80° C., they will still retain good rheological properties. As a consequence, the injectable products of the invention may be characterized by this parameter which, for more clarity, will be expressed by a percentage conservation of the modulus G′.

In the context of the invention, the “percentage of conservation of the modulus G′” corresponds to the ratio of the modulus G′ (in Pa), measured after storing the product at 80° C. for 96 hours, to the modulus G′ (in Pa) measured at time to, i.e. before the temperature is raised, advantageously measured at ambient temperature (e.g. 25° C.), multiplied by 100.

Preferably, the injectable products of the invention have a percentage of conservation of the modulus G′ of more than about 25%, 26% or even 27%, advantageously greater than or equal to about 28%, 29%, 30%, 35%, 40%, 45%, 50% or even 60%.

In other words, the present invention concerns implantable or injectable products, advantageously hyaluronic acid based products, having a value for the modulus G′, measured after storage for 96 hours at 80° C., greater than 27% or even greater than or equal to 28%, 29%, 30%, 35%, 40%, 45%, 50% or 60% of their initial value G′, advantageously measured at ambient temperature (e.g. 25° C.).

According to another embodiment, for a given initial G′ value, the products of the invention better resist high temperatures than the available products not prepared using the method of the invention. In other words, for a given value of G′, the percentage of conservation for a product of the invention will be greater than the one of a product prepared according to the prior art.

This test, known as the “forced heat degradation” test, can be used to:
extrapolate the physico-chemical behaviour of the products of the invention to being subjected to a lower temperature but over a longer period. Accelerated ageing is carried out because of the time/temperature equivalence. Thus, a parallel can be drawn with the durability of the products in the human body, at 37° C., over several months;
evaluate the resistance of the products in accordance with the invention to thermal degradation possibly linked to the preparation method, in particular to sterilization by heat treatment.

Another aspect of the invention concerns the size of the polymer networks, especially the networks of crosslinked hyaluronic acid, present in the products obtained in the frame of the invention.

As already mentioned, the crosslinking reaction allows forming networks of polymeric hyaluronic acid. The longer and/or more branched is the polymer, the larger the size of the network. The size of the networks of crosslinked polymer obtained in this manner is critical, in particular in view of the envisaged applications. Indeed, it is assumed that below a certain size, assessed to be 100 μm, the fragments of hyaluronic acid do not have a filling effect. Besides and as already mentioned, the fragments of hyaluronic acid are not adapted to be used as viscosupplementation agents. Furthermore, small sized networks of polymer (corresponding “soluble HA”) are dissolved more rapidly and are thus degraded in the organism. Thus, a filling or lubricating product comprising a large quantity of large-sized polymer networks will perform better and will last longer.

The evaluation of this parameter may in particular be carried out by means of a test consisting of carrying out an extraction of the sample (gel) with the aid of water, then passing the suspension of gel in water over a membrane (filter, sieve) with pores with an average pore diameter of about 100 μm. Details regarding the experimental protocol which may be carried out are presented in the “examples” section below.

Thus, the hyaluronic acid in the form of a small sized polymer network (less than about 100 μm) passes through the membrane and is located in the liquid aqueous phase, as is the free HA and/or the lidocaine added to the product. These various quantities (extracted HA corresponding to crosslinked HA in the form of a small sized network of polymer, free HA and/or lidocaine) are evaluated by measuring the total quantity of organic carbon (TOC) present in this phase. From this, the quantity of extracted HA can then be deduced using the following formula:

$$HA extract ( mg / g ) = \frac{( {[ TOC ]}_{Total} - {[ TOC ]}_{Lidocaine} ) \times 0.001 \times 401.3 \times {Q}_{total}}{168.2 \times PE}$$

in which:
[TOC]_{Total}: total TOC content of the extracted assay (mg/l)
401.3: molecular weight of HA (g/mol)
168.2: molecular weight of carbon in the HA (g/mol)
Qₜₒₜₐₗ: total extraction volume (g)=sample+water
PE: sample size=gel (g)
[TOC]_{lidocaine}: TOC content of lidocaine in the extracted assay (=0 when no lidocaine has been added), calculated using the formula below:

$$[ TOC ] Lidocaine ( mg / l ) = \frac{{[ Lidocaine ]}_{Extract} \times 168.2 \times 1000}{270.5}$$

in which:
168.2: molecular weight of carbon in lidocaine HCl (g/mol)
270.5: molecular weight of lidocaine HCl (g/mol)
[Lidocaine]Extract: Concentration of lidocaine HCl in the extract (mg/ml), determined by HPLC.

From this, the proportion of hyaluronic acid in the form of a network of crosslinked polymer with a size of less than about 100 μm (“HA soluble”) with respect to the total crosslinked hyaluronic acid present in the test product can be deduced using the following formula:

$$HA soluble ( \% ) = \frac{HA extract ( mg / g ) - HA free ( mg / g )}{{[ HA ]}_{total} ( mg / g ) - HA free ( mg / g )} \times 100$$

in which:
HA extract: calculated in accordance with the preceding paragraph
[HA]ₜₒₜₐₗ: total HA content in the gel (mg/g), determined by gravimetry.

Thus, and as supported by the results from the experimental section, the products of the invention are characterized in that the very large majority of the crosslinked hyaluronic acid they contain is in the form of polymer networks with a size of more than about 100 μm. In practice and to make it clearer, this characteristic is illustrated by the ratio between the hyaluronic acid present in the polymer networks with a size of less than about 100 μm and the total crosslinked hyaluronic acid present in the product. The person skilled in the art will have no difficulty in measuring this ratio in view to their general knowledge, the formulae set out above for calculations and based, for example, on the tests described in the experimental section.

The term “hyaluronic acid present in polymer networks of a size of less than about 100 μm” as used in the frame of the invention only designates the hyaluronic acid present in the form of polymer networks, and therefore does not include free hyaluronic acid, i.e. the non-crosslinked chains of hyaluronic acid added to the injectable product.

Preferably, the products of the invention have a quantity of hyaluronic acid in the form of polymer networks of a size of less than about 100 μm, with respect to the total quantity of crosslinked hyaluronic acid, of less than about 20%, advantageously less than or equal to about 19%, 18%, 17%, 16%, 15%, 10%, or even less than or equal to about 5%.

Thus, and advantageously, an implantable or injectable product of the invention has a proportion of crosslinked hyaluronic acid, present in the form of polymer networks with a size inferior to about 100 μm, of less than or equal to about 16%, 15%, 14%, 13%, 12%, 11%, 10% or even less than or equal to about 9%, 8%, 7%, 6% or even less than or equal to about 5%.

In other words, for a given value of G′, the proportion of crosslinked hyaluronic acid present in the form of polymer networks with a size inferior to about 100 μm will be less than the one of a product prepared according to the prior art.

The product in accordance with the invention may be introduced into devices which are suitable for intradermal injection, such as syringes, advantageously in a sterile form. Thus, the invention also concerns kits comprising at least one product in accordance with the invention and possibly a device suitable for intradermal injection.

In accordance with another aspect, the present invention concerns the use of these products, in particular with respect to their aesthetic and therapeutic applications.

Thus, and in accordance with a first aspect, the present invention concerns the use of a product of the invention for the prevention and/or the treatment, advantageously the filling of skin defects, in particular wrinkles and fine lines or scars. These defects may concern the face, but also any other part of the body.

These applications relate to both the field of aesthetic surgery as well as that of plastic surgery.

In these applications, the products of the invention may be administered in different ways, by different routes, in particular intradermally, hypodermically, subcutaneously or into the mucous membranes of the lips.

They may be recommended for various types of indications, listed here in a non-limiting manner:
volume corrections;
surface corrections (scars, etc);
correction of surface fine lines;
corrections of medium to deep wrinkles;
corrections of the eye contour (dark rings, folds, etc);
increasing the volume of the lips;
corrections of peri-oral wrinkles;
breast enhancement as an alternative to prostheses;
buttock sculpture.

The characteristics of the product to be administered, in particular in terms of its the strength to resist linear compression, as well as the route of administration may be determined by the skilled person skilled, in particular in the light of the envisaged indication.

Furthermore, the products in accordance with the invention have applications in the therapeutic field, for example.

In accordance with another aspect, the present invention concerns the use of such a product for the prevention and/or treatment of joint problems, in particular osteoarthritis.

In this context, the product supplements endogenous hyaluronic acid in its role as a lubricant and is termed a viscosupplementation agent.

The product of the invention may have other applications necessitating the filling of cavities or the generation of volume, for example in order to exert a pressure or to space tissues apart. The field of otorhinolaryngology may be mentioned, in particular the repair of the vocal cords, or in urology or ophthalmology. Another application concerns the use of this type of products to isolate organs, in particular in the case of cancer treatment using radiation, e.g. by radiotherapy. Thus, the product of the invention may be implanted or injected between the prostate and the rectum in the case of the treatment of colorectal cancer. In all of these applications, the product in accordance with the invention is used as a tissue spacer.

For purposes of the present invention, it should be noted that to provide a more concise description, some of the quantitative expressions given herein are not qualified with the term “about.” It is understood, however, that whether the term “about” is used explicitly or not, all numbers are to be understood as being modified by the term “about,” whether or not so expressly stated. Similarly, a range given of “about 1% to 10%” is intended to have the term “about” modifying both the 1% and the 10% endpoints.

Unless otherwise indicated expressly, steps may be performed in any order, without limitation.

### EXAMPLES

The invention and the advantages resulting therefrom will become clearer from the following exemplary embodiments, which relate to hyaluronic acid (HA) but which, however, are not in any way limiting in scope.

### I—Inventive methods and products

### Example 1: Preparation of Products

Injectable products were prepared from the reaction mixtures indicated in Table 1 below:

TABLE 1

| | |
|---|---|
| Product A | Product B |

| | |
|---|---|
| Average molecular | 1.5 |

| | | |
|---|---|---|
| weight of NaHA (MDa) | | |
| Molar Ratio crosslinking | 4.3 | 8.2 |
| agent/disaccharides (%) | | |

| | |
|---|---|
| HA Concentration in the | 125 |

reaction mixture (mg/g)

The preparation method was as follows:

A mass of sodium hyaluronate (NaHA) fibers with an average molecular weight of 1.5 MDa (1.5×10⁶ Da), necessary for obtaining the concentration indicated in Table 1, was weighed out and placed in a container.

A solution of crosslinking agent composed of distilled water, sodium hydroxide and 1,4-butanediol diglycidyl ether (BDDE, CAS number: 2425-79-8) in a concentration necessary for obtaining the ratio indicated in Table 1, was added to the NaHA fibres. The mixture was homogenized.

Once the reaction medium had been mixed, the container was placed in a refrigerated vessel at a temperature in the range 0° C. to 10° C. for several days.

At the end of this step, a gel was obtained.

The gel so obtained was hydrated in order to obtain the desired concentration of hyaluronic acid at a physiological pH (in the range 6 to 8). The hydration bath was composed of phosphate buffer and 1N hydrochloric acid. The proportions were adapted as a function of the concentration of HA during crosslinking and that which was to be obtained. The gel was cut into pieces and then introduced into the hydration bath.

This hydrated gel was then purified by washing with phosphate buffer in order to eliminate residual crosslinking agent.

Free sodium hyaluronate (i.e. not crosslinked) with an average molecular weight of 1.5 MDa was added to the purified gel, as a function of the desired final concentration of free HA (see Table 2 below).

A 10% (w/w) solution of lidocaine was added to the injectable product in order to obtain a final concentration of 3 mg/g of lidocaine in the gel.

The concentration of crosslinked HA was adjusted by adding phosphate buffer (see Table 2 below).

The mixture was degassed.

Once degassing was complete, syringes were filled then sterilized.

The injectable products obtained are described in Table 2 below:

TABLE 2

| | Product A | Product B |
|---|---|---|
| Final concentration of | 18 | 22 |
| modified HA (mg/g) | | |
| Final concentration of | 2 | 1 |
| free HA (mg/g) | | |

### Example 2: Mechanical and Rheological Properties of Products Obtained

The modulus G′, the strength to resist linear compression (F) and the degree of modification (MoD) of the products prepared in Example 1 were measured.

The modulus G′ was determined with the aid of a Kinexus Pro rheometer using a 40 mm plate/plate geometry, with a 250 μm gap, at ambient temperature (25° C.). Pre-shearing and a rest period were applied to the sample in order to erase its rheological memory then the linear viscoelastic domain was determined by an amplitude sweep at a fixed frequency of 1 Hz. The modulus G′ was determined from the curve obtained in the linear viscoelastic domain where the stress and the displacement are in a linear relationship.

The strength to resist linear compression (F) was determined with the aid of a rheometer with a 20 mm plate/plate geometry with a gap varying from 2.5 mm to 0.9 mm at a speed of 0.1 mm/sec, at ambient temperature (25° C.).

The degree of modification (MoD) was determined using Kenne et al.'s method (Carbohydrate Polymers; 91(1): 410-4182, 2013). In brief, the product to be analysed was washed in a sodium chloride solution then filtered under vacuum using a 0.22 μm filter before being digested with chondroitinase AC at 37° C. overnight. The number of moles of crosslinking agent bound to the molecules of hyaluronic acid and the number of moles of hyaluronic acid effectively crosslinked were determined by ²D NMR, with integration of the signal corresponding to residues of crosslinking agent and of the signal corresponding to the N-acetyl hyaluronic acid, after correction for the number of protons responsible for each signal.

The results obtained are indicated in Table 3 below:

TABLE 3

| | Product A | Product B |
|---|---|---|
| Rheology G′(Pa) | 124 | 439 |
| Strength to resist linear | 0.55 | 0.93 |
| compression F (N) | | |
| Degree of modification MoD (%) | 3.0 | 5.39 |
| F/MoD ratio (absolute value) | 0.18 | 0.17 |

### Example 3: Thermal Stability of Products Obtained

In order to estimate the thermal stability of the injectable products obtained, the gels A and B described above were subjected to a high temperature for a short period.

In brief, samples of the products to be analysed were placed at 80° C. for several days, for example 1, 2, 3 or 4 days. Thereafter, samples were removed then placed at ambient temperature until they had cooled to this temperature. Once at ambient temperature, the elastic modulus G′ was measured in accordance with the method described in Example 2.

The results obtained are indicated in Table 4 below:

TABLE 4

| | Product A | Product B |
|---|---|---|
| % conservation of G′ | 32 | 61 |
| (=G′_{96 h}/G′ₜ₀ × 100) | | |

### Example 4: Estimation of In Vivo Performance of Products Obtained

The percentage of soluble sodium hyaluronate (HA soluble) in the crosslinked fraction of the HA gels obtained was estimated after extraction of the sample in water and filtration over a 100 μm sieve. In fact, below this size, it was assumed that HA, whether crosslinked or not, has no filling effect and is rapidly degraded in the organism.

HA soluble was determined by calculation after assaying the Total Organic Carbon (TOC).

If the products obtained contained lidocaine, the TOC provided by the lidocaine was taken into account by assaying the lidocaine present in the test sample.

In the case in which free HA was added to the product obtained in order to facilitate injection and not for filling, the quantity of free HA added to the product was deducted from the extracted HA.

### Extraction Protocol:

In brief, 1 gram of product to be analysed (sample) was mixed with 100 g of purified water. The mixture was stirred for several hours at ambient temperature.

### Filtration Protocol:

The mixture was then filtered over a cloth the pores of which had an average diameter of 100 μm.

Calculation of HA Extracted from Product:
a—TOC Content of Lidocaine:

$$[ TOC ] Lidocaine ( mg / l ) = \frac{{[ Lidocaine ]}_{sample} \times 168.2 \times 1000}{270.5}$$

in which:
168.2: molecular weight of carbon in lidocaine HCl (g/mol)
270.5: molecular weight of lidocaine HCl (g/mol)
[Lidocaine]Extract: concentration of lidocaine HCl in sample (mg/ml), determined by HPLC
   b—HA Extracted, in mg/g of Product Obtained

The quantity of total organic carbon (TOC) present in the liquid phase obtained (hereinafter the “HA extract”) was measured:

$$HA extract ( mg / g ) = \frac{( {[ TOC ]}_{Total} - {[ TOC ]}_{Lidocaine} ) \times 0.001 \times 401.3 \times {Q}_{total}}{168.2 \times PE}$$

in which:
[COT]_{Total}: total TOC in test sample (mg/l)
[COT]_{lidocaine}: TOC content in lidocaine assayed in sample (see a. above)
401.3: molecular weight of HA (g/mol)
168.2: molecular weight of carbon in the HA (g/mol)
Qₜₒₜₐₗ: total extraction volume (sample+water)
PE: sample size (g)
   c. Soluble HA as % of HA in Product Obtained

$$HA soluble ( \% ) = \frac{HA extract ( mg / g ) - HA free ( mg / g )}{{[ HA ]}_{total} ( mg / g ) - HA free ( mg / g )} \times 100$$

in which:
HA extract: see b. above
[HA]total: total HA content in sample (mg/g), determined by gravimetry.

The results obtained are indicated in Table 5 below:

TABLE 5

| | Product A | Product B |
|---|---|---|
| Soluble HA (%) in HA | 15 | 4 |
| cross-linked fraction | | |

It can be seen that the quantity of HA said to be soluble (in the form of a network of polymer with a size of less than 100 μm) in the products obtained is low. As a consequence, in the products of the invention, the majority of the chains of crosslinked hyaluronic acid are the form of large-sized networks of polymer, so that effective filling and long-lasting performance are obtained.

### II—Comparison of the Products of the Invention to Commercial Products

The characteristics of the products obtained by the methods of the invention have been compared to commercial products known to be crosslinked with BDDE at high temperature (50° C.). The data are shown in Table 6 below:

TABLE 6

| | | | | | | | Thermal |
|---|---|---|---|---|---|---|---|
| | | | Total HA | | | Ratio | stress test |
| | | Rheology- | content | Modification | Compression | Compression/ | 96 h-80° C. |
| Product | Name | G′ (Pa) | (mg/g) | degree (%) | test (N) | MoD | G′/G′ₜ₀ |
| Products | A | 124 | 20 | 3 | 0.55 | 0.18 | 0.32 |
| according to | B | 439 | 23 | 5.4 | 0.93 | 0.17 | 0.61 |
| the invention | | | | | | | |
| Restylane | Restylane | 524 | 20 | 2.1 | 0.25 | 0.12 | 0.26 |
| Emervel | Classic | 97 | 20 | 5.5 | 0.56 | 0.10 | 0.10 |
| | Deep | 280 | 20 | 7.9 | 0.49 | 0.06 | 0.16 |
| Juvederm | Voluma | 246 | 20 | 6.0 | 0.88 | 0.15 | 0.27 |
| | Ultra 3 | 160 | 24 | 6.5 | 0.76 | 0.12 | 0.12 |

### III—Efficacy of the Products of the Invention as Fillers

A prospective multicentre clinical study has been launched to evaluate the safety and efficacy of the products according to the invention in the treatment of pathological facial lipoatrophy.

Both products A and B, with a majority of B, as disclosed in part I of the examples were injected in two sessions 14 days apart (Total volume 7 ml for B, 2 ml for A) in patients for correction of facial lipoatrophy by mid face augmentation. Patients were followed up for up to 12 months. The results were evaluated with the Global Aesthetic Improvement Scale, GAIS.

The study has allowed to demonstrate that even after 12 months, 100% of the patients were still responder to the treatment.

1. A method of preparing a product containing a crosslinked polymer, wherein the crosslinking step consists in incubating in a reaction mixture the polymer, or one of its physiologically acceptable salts, with a crosslinking agent having at least two epoxy functions, at a temperature of more than 0° C. and less than 10° C., advantageously between 1° C. and 9° C.

2. The method of claim 1, wherein the polymer is selected from the glycosaminoglycan group, advantageously hyaluronic acid or sodium hyaluronate.

3. The method of claim 1, wherein the crosslinking agent is chosen from 1,4-butanediol diglycidyl ether (BDDE), ethylene glycol diglycidyl ether, diethylene glycol diglycidyl ether, or tetraethylene glycol diglycidyl ether, advantageously BDDE.

4. The method of claim 1, wherein the crosslinking step is carried out for a period of at least 48 hours, advantageously at least 7 days.

5. The method of claim 2, wherein the molar ratio between the crosslinking agent and the disaccharide units of the polymer is less than or equal to 10%, and advantageously greater than or equal to 0.5%.

6. The method of claim 1, wherein at the end of the crosslinking step, the reaction mixture undergoes at least one of the following treatments: neutralization, dilution, purification, sterilization.

7. An implantable or injectable product obtained by the method of of claim 1, advantageously a hyaluronic acid based product.

8. The implantable or injectable product of claim 7, having:
a degree of modification (MoD) of less than 10%, advantageously between 0.5% and 6%; and
a strength to resist linear compression (F) between 0.2 N and 1.2 N, or even between 0.35 N and 1 N, when it is compressed at 25° C. using a rheometer with a 20 mm plate/plate geometry and a gap varying from 2.5 mm to 0.9 mm, at a speed of 0.1 mm/sec.

9. The implantable or injectable product of claim 8, wherein it has a ratio F/MOD greater than 0.15.

10. The implantable or injectable product of claim 7, having a value for the modulus G′, measured after storage for 96 hours at 80° C., greater than 27%, even greater than or equal to 30%, 40%, 50% or 60% of its initial G′ value.

11. The implantable or injectable product claim 7, having a proportion of crosslinked hyaluronic acid, present in the form of networks of size inferior to 100 μm, of less than 20%, 15%, 10% or even less than 5%.

12. The implantable or injectable product of claim 7, containing crosslinked hyaluronic acid representing 1 to 40 mg/g of product, advantageously 10 to 25 mg/g of product.

13. The implantable or injectable product of claim 7, containing unmodified hyaluronic acid, advantageously representing 0.5 to 5 mg/g of product, more advantageously 1 to 3 mg/g of product.

14. The implantable or injectable product of claim 7, further comprising an anaesthetic agent, advantageously lidocaine.

15. A kit or a syringe comprising the implantable or injectable product of claim 7.

16. A method of treating skin defects, e.g. fine lines, wrinkles and scars, comprising administering the implantable or injectable product of claim 7.

17. The method of claim 16, wherein the product is administered intradermally, hypodermically or subcutaneously.

18. A method of joint problems, advantageously osteoarthritis, comprising administering the implantable or injectable product of claim 7.

19. A method comprising using the product of claim 7 as a tissue spacer.
